SEQUENCE LISTING (4 sequences)

SEQ ID NO: 1 - DNA, 2166 bp, Homo sapiens
atgaaagagatgtctgcaaacaccgtgctggacagccagcgtcaacaaaagcattatggaattacctccccaattagtttggcatctcctaaagaaattgatcatatttacacacagaaattaattgacgccatgaaaccatttggagtgtttgaagatgaggaagaattgaaccacaggctggtggttcttggtaaattgaacaatttagtaaaagaatggatttctgatgtcagcgagagtaagaacctcccaccttctgttgtggctactgttggtggtaaaattttcacatttggatcctataggcttggagtacacaccaaaggagctgacattgatgcactttgtgtagctccaagacatgtggaaagatctgatttttttcagtctttttttgaaaaattgaaacatcaagatggcattagaaacttaagagctgtagaagatgcctttgtacctgttataaaatttgaatttgatggtattgaaattgatctagtctttgcaagactggcaatacaaaccatatcagataatttagatctaagagacgactctcgcctgagaagccttgatataaggtgtattcgcagcttaaatggttgtagagttactgatgaaattttgcatttagtgccaaataaagaaacttttagactcaccctaagagctgtcaaattatgggcaaaacgacgtggtatttattccaacatgctaggattccttggtggtgtctcctgggcaatgctagttgcaagaacttgccaattgtatccaaatgcagcagcatctactttagttcataagttctttttagttttttccaagtgggaatggccaaatcctgtgctgctgaagcaaccagaagaaagcaatttgaatttgcctgtctgggatcctcgggtaaatccatcagataggtatcatctcatgcccataatcacccctgcctacccacaacagaattctacgtataatgtgtccacatcaactcgaacagtaatggtagaagaatttaaacaaggtcttgcagtcacagatgaaattcttcaaggaaagtcagattggtccaaactacttgagccaccgaatttctttcaaaagtataggtacgttggattagtagaatctaaaatccgtgtacttgttggaaacttggaacggaatgaatttattactcttgcccatgtgaatccccagtcattcccagggaataaggaacatcataaagacaacaattacgtatcaatgtggttccttgggataatttttcggagagtagaaaatgcagaaagtgtcaacatagacttgacatatgatatacagtcatttactgatacagtgtacagacaggcaaacaatataaatatgctaaaggagggaatgaaaattgaagcaactcatgtaaagaaaaaacaacttcaccactaccttcctgcagaaattcttcaaaagaagaaaaaggtgagtcaaagtctctctgatgtcaatcgaagctcgggcggacttcaatccaaaagattgtctctggatagcagttgtctggatagctccagagacactgataatggaacaccttttaattctccagcgtccaagtctgatagcccttctgtaggagaaacagaaaggaatagtgctgagcctgctgctgtaattgtggagaagccactgagtgtaccaccagcccaaggactttccattccagtgattggcgcaaaagttgactctacagtaaaaactgtatcaccccccactgtgtgtaccattcctaccgtagtaggacgaaatgtcattcctagaatcacaacacctcacaaccctgcccagggacaaccgcatctgaatggaatgtcaaatataactaagactgttacacctaagagatcccattccccatccatagatgggactcctaagaggttgaaagacgtagaaaagtttattcgacttgaatcaacatttaaggacccccgcactgctgaagaaagaaaaagaaaatcagtggatgccattggaggagaatctatgcctattccaactattgatacatcacgcaaaaagagactacccagtaaagaactaccagattcatcatctccagttccagcaaacaacatccgtgtcatcaaaaattccattcgactgacccttaatcggtaa

SEQ ID NO: 2 - protein, 722 aa, Homo sapiens
MKEMSANTVLDSQRQQKHYGITSPISLASPKEIDHIYTQKLIDAMKPFGVFEDEEELNHRLVVLGKLNNLVKEWISDVSESKNLPPSVVATVGGKIFTFGSYRLGVHTKGADIDALCVAPRHVERSDFFQSFFEKLKHQDGIRNLRAVEDAFVPVIKFEFDGIEIDLVFARLAIQTISDNLDLRDDSRLRSLDIRCIRSLNGCRVTDEILHLVPNKETFRLTLRAVKLWAKRRGIYSNMLGFLGGVSWAMLVARTCQLYPNAAASTLVHKFFLVFSKWEWPNPVLLKQPEESNLNLPVWDPRVNPSDRYHLMPIITPAYPQQNSTYNVSTSTRTVMVEEFKQGLAVTDEILQGKSDWSKLLEPPNFFQKYRYVGLVESKIRVLVGNLERNEFITLAHVNPQSFPGNKEHHKDNNYVSMWFLGIIFRRVENAESVNIDLTYDIQSFTDTVYRQANNINMLKEGMKIEATHVKKKQLHHYLPAEILQKKKKVSQSLSDVNRSSGGLQSKRLSLDSSCLDSSRDTDNGTPFNSPASKSDSPSVGETERNSAEPAAVIVEKPLSVPPAQGLSIPVIGAKVDSTVKTVSPPTVCTIPTVVGRNVIPRITTPHNPAQGQPHLNGMSNITKTVTPKRSHSPSIDGTPKRLKDVEKFIRLESTFKDPRTAEERKRKSVDAIGGESMPIPTIDTSRKKRLPSKELPDSSSPVPANNIRVIKNSIRLTLNRZ

SEQ ID NO: 3 - DNA, 2211 bp, Homo sapiens
atgaaagagatgtctgcaaacaccgtgctggacagccagcgtcaacaaaagcattatggaattacctccccaattagtttggcatctcctaaagaaattgatcatatttacacacagaaattaattgacgccatgaaaccatttggagtgtttgaagatgaggaagaattgaaccacaggctggtggttcttggtaaattgaacaatttagtaaaagaatggatttctgatgtcagcgagagtaagaacctcccaccttctgttgtggctactgttggtggtaaaattttcacatttggatcctataggcttggagtacacaccaaaggagctgacattgatgcactttgtgtagctccaagacatgtggaaagatctgatttttttcagtctttttttgaaaaattgaaacatcaagatggcattagaaacttaagagctgtagaagatgcctttgtacctgttataaaatttgaatttgatggtattgaaattgatctagtctttgcaagactggcaatacaaaccatatcagataatttagatctaagagacgactctcgcctgagaagccttgatataaggtgtattcgcagcttaaatggttgtagagttactgatgaaattttgcatttagtgccaaataaagaaacttttagactcaccctaagagctgtcaaattatgggcaaaacgacgtggtatttattccaacatgctaggattccttggtggtgtctcctgggcaatgctagttgcaagaacttgccaattgtatccaaatgcagcagcatctactttagttcataagttctttttagttttttccaagtgggaatggccaaatcctgtgctgctgaagcaaccagaagaaagcaatttgaatttgcctgtctgggatcctcgggtaaatccatcagataggtatcatctcatgcccataatcacccctgcctacccacaacagaattctacgtataatgtgtccacatcaactcgaacagtaatggtagaagaatttaaacaaggtcttgcagtcacagatgaaattcttcaaggaaagtcagattggtccaaactacttgagccaccgaatttctttcaaaagtatagacattatatagtattgactgccagcgcatcaacagaagaaaaccatctagagtgggttggattagtagaatctaaaatccgtgtacttgttggaaacttggaacggaatgaatttattactcttgcccatgtgaatccccagtcattcccagggaataaggaacatcataaagacaacaattacgtatcaatgtggttccttgggataatttttcggagagtagaaaatgcagaaagtgtcaacatagacttgacatatgatatacagtcatttactgatacagtgtacagacaggcaaacaatataaatatgctaaaggagggaatgaaaattgaagcaactcatgtaaagaaaaaacaacttcaccactaccttcctgcagaaattcttcaaaagaagaaaaagcaaagtctctctgatgtcaatcgaagctcgggcggacttcaatccaaaagattgtctctggatagcagttgtctggatagctccagagacactgataatggaacaccttttaattctccagcgtccaagtctgatagcccttctgtaggagaaacagaaaggaatagtgctgagcctgctgctgtaattgtggagaagccactgagtgtaccaccagcccaaggactttccattccagtgattggcgcaaaagttgactctacagtaaaaactgtatcaccccccactgtgtgtaccattcctaccgtagtaggacgaaatgtcattcctagaatcacaacacctcacaaccctgcccagggacaaccgcatctgaatggaatgtcaaatataactaagactgttacacctaagagatcccattccccatccatagatgggactcctaagaggttgaaagacgtagaaaagtttattcgacttgaatcaacatttaaggacccccgcactgctgaagaaagaaaaagaaaatcagtggatgccattggaggagaatctatgcctattccaactattgatacatcacgcaaaaagagactacccagtaaagaactaccagattcatcatctccagttccagcaaacaacatccgtgtcatcaaaaattccattcgactgacccttaatcggtaa

SEQ ID NO: 4 - protein, 736 aa, Homo sapiens
MKEMSANTVLDSQRQQKHYGITSPISLASPKEIDHIYTQKLIDAMKPFGVFEDEEELNHRLVVLGKLNNLVKEWISDVSESKNLPPSVVATVGGKIFTFGSYRLGVHTKGADIDALCVAPRHVERSDFFQSFFEKLKHQDGIRNLRAVEDAFVPVIKFEFDGIEIDLVFARLAIQTISDNLDLRDDSRLRSLDIRCIRSLNGCRVTDEILHLVPNKETFRLTLRAVKLWAKRRGIYSNMLGFLGGVSWAMLVARTCQLYPNAAASTLVHKFFLVFSKWEWPNPVLLKQPEESNLNLPVWDPRVNPSDRYHLMPIITPAYPQQNSTYNVSTSTRTVMVEEFKQGLAVTDEILQGKSDWSKLLEPPNFFQKYRHYIVLTASASTEENHLEWVGLVESKIRVLVGNLERNEFITLAHVNPQSFPGNKEHHKDNNYVSMWFLGIIFRRVENAESVNIDLTYDIQSFTDTVYRQANNINMLKEGMKIEATHVKKKQLHHYLPAEILQKKKKQSLSDVNRSSGGLQSKRLSLDSSCLDSSRDTDNGTPFNSPASKSDSPSVGETERNSAEPAAVIVEKPLSVPPAQGLSIPVIGAKVDSTVKTVSPPTVCTIPTVVGRNVIPRITTPHNPAQGQPHLNGMSNITKTVTPKRSHSPSIDGTPKRLKDVEKFIRLESTFKDPRTAEERKRKSVDAIGGESMPIPTIDTSRKKRLPSKELPDSSSPVPANNIRVIKNSIRLTLNR

(19) United States
(12) Patent Application Publication
(10) Pub. No.: US 2004/0161762 A1
(43) Pub. Date: Aug. 19, 2004

(54) Novel human poly(a)polymerase gamma (pap gamma)

(76) Inventors: Anders Virtanen, Uppsala (SE); Helena Nordvarg, Uppsala (SE); Christina Kyriakopolou, Brussels (BE)

Correspondence Address: BROWDY AND NEIMARK, P.L.L.C., 624 NINTH STREET, NW, SUITE 300, WASHINGTON, DC, 20001-5303, US

(21) Appl. No.: 10/470,534

(22) PCT Filed: Feb. 8, 2002
(86) PCT No.: PCT/SE02/00216

(30) Foreign Application Priority Data
Feb. 9, 2001 (SE) 0100412-6

(51) Int. Cl. C12Q 1/68; C07H 21/04; C12N 9/22

(52) U.S. Cl. 435/6; 435/69.1; 435/199; 435/320.1; 435/325; 536/23.2

(57) ABSTRACT

The present invention relates to a novel human poly(A)polymerase enzyme (PAP), its nucleic and amino acid sequence and use thereof as well as an antibody against the novel enzyme and use thereof. The novel enzyme is named PAP gamma and is not related to the previously known PAPs.

### FIELD OF THE INVENTION

The present invention relates to a novel human poly(A) polymerase enzyme (PAP), its nucleic and amino acid sequence and use thereof as well as an antibody against the novel enzyme and use thereof.

### BACKGROUND OF THE INVENTION

The majority of mammalian messenger RNAs end with a 200-250 adenosine residues tail at their 340 ends. The function of the poly(A) tail is not fully understood but detailed studies have highlighted its role in regulating gene expression via translational regulation and mRNA stability control (reviewed in (Mitchell and Tollervey, 2000; Sachs and Varani, 2000).

The mRNA's poly(A) tail is added post-transcriptionally to the pre-mRNA and the biochemistry of mammalian nuclear polyadenylation has been extensively studied (reviewed in (Wahle and Ruegsegger, 1999; Zhao et al., 1999)). The reaction is a multi-step and multi-component reaction which proceeds through two independent steps; RNA cleavage and adenosine addition. Both reactions are dependent on a highly conserved sequence-element, the hexanucleotide AAUAAA. At least six trans-acting protein factors are required for the reaction in vivo.

Poly(A) polymerase (PAP), one of these factors, is the enzyme responsible for poly(A) tail addition. Mammalian PAP has been identified in and cloned from several species among them human, mouse and bovine (Raabe et al., 1991; Thuresson et al., 1994; Wahle et al., 1991; Zhao and Manley, 1996). Experimental data show that the degree of phosphorylation of PAP varies during Xenopus oocyte maturation (Ballantyne et al., 1995) and early development; in these cases the key regulatory mechanism in gene expression is the selective translational activation of maternal mRNAs, which get a long poly(A) tail and recruit to the polyribosomes. PAP phosphorylation varies as well as during the cell cycle (Colgan and Manley, 1997; Colgan et al., 1996). PAP is hyperphosphorylated in HeLa cells arrested in mitosis and the enzyme's activity is inhibited. It is also known that cells entering mitosis show a general repression of RNA and protein synthesis, and inhibition of PAP activity could contribute to this in several ways.

Multiple forms of poly(A) polymerase (PAP) have been identified in mammalian cell lines and tissues (e.g. (Thuresson et al., 1994) reviewed in (Wahle and Ruegsegger, 1999; Zhao et al., 1999)). HeLa cell nuclear extracts contain several isoforms of PAP, having apparent molecular weights of 90, 100, 106 kDa, as identified by immunoblotting. It has previously been shown that the 106 kDa form is post-translationally modified by phosphorylation (Thuresson et al., 1994). Several forms of PAP are generated by alternative splicing since a series of alternatively spliced mRNAs have been identified (Raabe et al., 1991; Thuresson et al., 1994; Wahle et al., 1991; Zhao and Manley, 1996). The combination alternative splicing and post-translational modifications arises the possibility of a very complex pattern of PAP isoforms. The identification of multiple PAP isoforms and their functional significance still remains an open question. However, since PAP participates in a whole set of different reactions (e.g. RNA cleavage, AAUAAA dependent and independent adenosine addition), at different subcellular locations (nucleus, cytoplasm), it seems reasonable to hypothesize that different PAPs are responsible for different functions in vivo. It is known that poly(A) polymerase is associated with several diseases, for example breast cancer (Scorilas et al., 1998, Scorilas et al 2000).

### SUMMARY OF THE INVENTION

The present inventors have managed to biochemically separate and purify PAPs representing each of the native isoforms from HeLa cells. A functional comparison using the specific (CPSF and hexanucleotide—AAUAAA dependent) and non-specific (CPSF independent) in vitro polyadenylation assays revealed differences between the isoforms. These differences correlate to the stability of the CPSF/PAP/RNA complex. By RT-PCR five alternative spliced forms of PAP mRNAs were identified. These forms are alternative spliced variants of the previously cloned human and bovine PAP. These forms differ in their carboxy-terminal ends due to alternative splicing and result in carboxy-terminal PAP isoforms.

Surprisingly, one of the presumed isoforms (90 kDa) turned out to be a novel human PAP enzyme encoded by a distinct gene, not linked to the previously identified human PAP gene (Kyriakopoulou et al. 2001).

The novel PAP has been named PAPγ by the Human Gene Nomenclature Committee to distinguish it from the previously known PAP which is encoded by a gene now renamed to PAPOLA. PAPγ is encoded by a unique gene named PAPOLG and is not related to the previously identified mammalian PAPs. The nucleotide sequence and the deduced amino acid sequence are provided in the Sequence Listings. PAPγ shares some nucleotide sequence homology with the previously identified human PAP in its N-terminal part (the first 500 aminoacids), approximately 75%. The C-terminal part, starting from this position is unique for the novel PAPγ with the exception of the last 18 amino acids which are shared between the two forms of human PAP encoded by the genes PAPOLA and PAPOLG.

Thus, the invention relates to an amino acid sequence for poly(A) polymerase γ (PAPγ) according to Sequence Listing Id No 2 and or Id No 4 and/or functional parts thereof having poly(A) polymerase (PAP) activity as well as essentially homologous, such as 90% homologous, variants of said sequence.

The invention also relates to a nucleic acid sequence according to Sequence Listing Id No 1 and/or Id No 3 encoding the amino acid sequence according to claim 1 and variants of said nucleic acid sequence due to the degeneracy of the genetic code.

Moreover, the invention relates to a vector comprising the above nucleic acid sequence as well as a host cell comprising the vector and other necessary elements for expression of the nucleic acid sequence encoding PAPγ.

Furthermore, the invention relates to a method for production of recombinant PAPγ, comprising cultivating the above host cell in a suitable cultivation media; and recovering PAPγ from said media.

The invention also relates to use of the above nucleic acid sequence or portions thereof for detection of PAPγ related diseases or disorders. In practice, the nucleic acid sequence or portions thereof will, for example, be used as a hybridisation probe for detection of corresponding sequences in patient samples. Preferably, a PAPγ specific part of the sequence is used.

The invention also relates to an antibody against PAPγ which is selective for PAPγ and does not react with other PAPs. This means that the antibody is directed against an epitope present in the C-terminal part of PAPγ. Polyclonal or monoclonal or fragments of antibodies produced by conventional methods are contemplated. An exemplifying antibody is described in the detailed section below. The antibody according to invention may be used for detection of PAP related diseases, such as different forms of cancer, for example breast cancer.

Finally, the invention relates to a reagent comprising PAPγ according to the invention. The reagent is intended for synthesizing and modifying RNA or as a target for the development of novel pharmaceutical drugs that either perturb or do not affect PAPγ activity. Preferably the drug target is derived from the unique C-terminal part of PAPγ.

### BRIEF DESCRIPTION OF THE DRAWINGS

FIG. 1 is a demonstration of poly(A) polymerase activity by specific incorporation of rATP. FIG. 1 shows polymerization activity of recombinant full length PAPγ in the presence of different nucleotide analogues.

FIG. 2 shows specific polyadenylation activity of PAPγ according to the invention.

FIG. 3 shows recognition of the native PAP isoforms in HeLa cell nuclear extracts, by different antibodies run on a 6% SDS polyacrylamide gel.

### BRIEF DESCRIPTION OF THE SEQUENCE LISTINGS

Sequence ID NO 1: DNA sequence of the novel PAPγ according to the invention.

Sequence ID NO 2: Amino acid sequence encoded by the DNA sequence according to SEQ ID NO 1.

Sequence ID NO 3: DNA sequence of a homologous variant of the novel PAPγ according to the invention.

Sequence ID NO 4: Amino acid sequence encoded by the DNA sequence according to SEQ ID NO 3.

### DETAILED DESCRIPTION OF THE INVENTION

The novel PAPγ was cloned and recombinant proteins were expressed in a prokaryotic expression system. Procedure for cloning and purification are found below. Initial kinetic analysis revealed that the new human PAPγ is equally good or better compared to the human PAPs encoded by the PAPOLA gene. All nucleotide and amino acid numbers below refer to SEQ Id No 3 and 4, respectively, as these are the preferred ones.

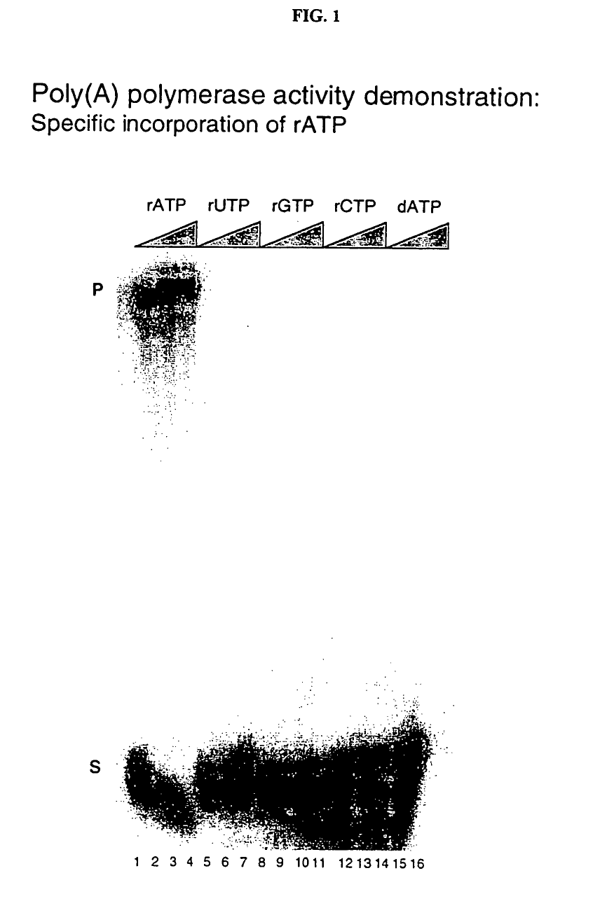

In FIG. 1 it is shown that the recombinant PAPγ has high specificity for synthesing poly(A) tails, using the non-specific assay in the presence of Mn(II). The reaction mixture contains in final concentrations: 100 mM Tris/HCl buffer pH 8.6 (RT), 40 mM KCl, 40 μM EDTA, 10% glycerol, 1.0 mM DTT, 0.9 units Rnasin (ribonuclease inhibitor), 0.1% NP-40, 0.5 mM MnCl₂, 0.5 mg/ml BSA and a mixture of cold and radiolabeled RNA substrate OligoA(15) (330 fmoles); PAP is added to the reaction at 3 different amounts of 17.5, 35, and 70 ng, respectively. The reactions proceed at 30 min, 37° C. The reaction is stopped by Proteinase K buffer, the RNA is extracted by phenol:chloroform and it is run in a 16% acrylamide: bis-acrylamide (19:1)-7 M urea gel. The gel is exposed overnight to a phosphoimager screen and further analyzed. The lanes represent the following: lane 1: negative control (no PAP), lanes 2-4: 0.5 mM rATP, lanes 5-7: 0.5 mM rUTP, lanes 8-10: 0.5 mM rGTP, lanes 11-13: 0.5 mM CTP and lanes 14-16: 0.5 mM dATP.

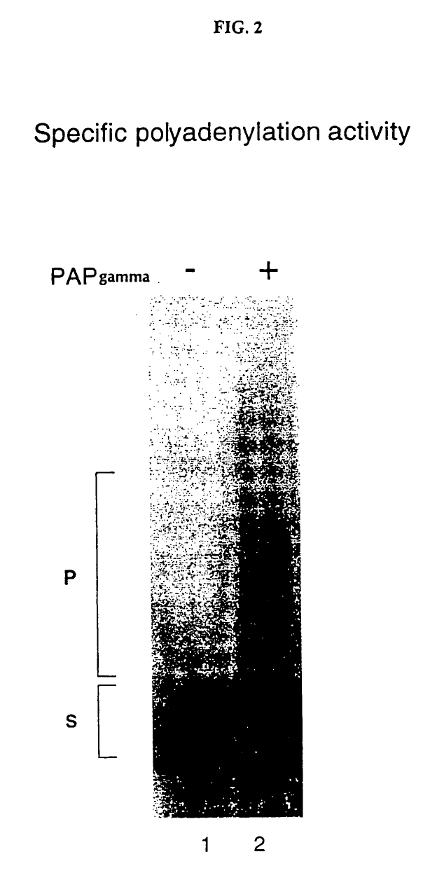

In FIG. 2 it is shown that PAPγ also functions in the AAUAAA and CPSF dependent assay. This data provides conclusive proof that poly(A) polymerase activity is associated with PAPγ. The reaction mixture contains in final concentrations: 100 mM Tris/HCl buffer pH 8.3 (RT), 40 mM KCl, 40 μM EDTA, 9.6% glycerol, 0.24 mM DTT, 0.9 units Rnasin (ribonuclease inhibitor), 0.01% NP-40, 0.72 mM MgCl2, 0.2 mg/ml BSA, 1 mM ATP, 2.5% PVA, 20 mM creatinine phosphate and a mixture radiolabeled RNA substrate L3(53) (70 fmoles in total). CPSF (partially purified from calf thymus) 3 μl, and PAP 50 ng are added to the reaction. The reactions proceed at 15-30 min., 30° C. The reaction is stopped by proteinase K buffer, the RNA is extracted by phenol:chloroformand and it is run in a 10% acrylamide:bis-acrylamide(19:1)-7 M Urea gel. The gel is exposed overnight to a phosphoimager screen and further analyzed. The lanes represent the following: lane 1: negative control (no PAP), lane 2: PAP+CPSF.

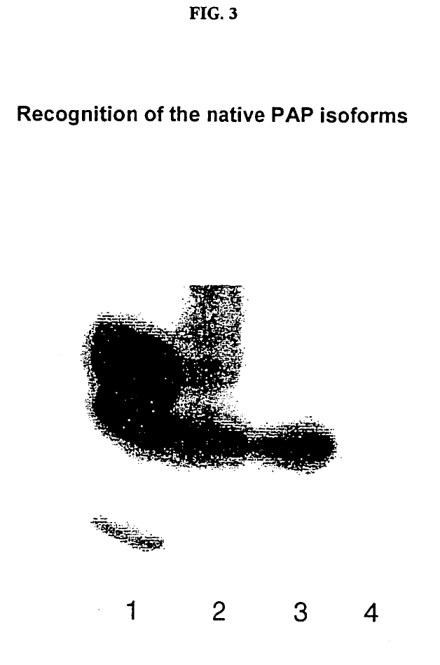

The development of specific polyclonal sera against the unique C-terminal part of the new enabled detection of PAPγ in HeLa nuclear extracts (FIG. 3). The proteins from the SDS gel are transferred to ImmobilonP membranes and immunostaining and visualization is done by ECLplus reagents. The lanes represent the following: lane 1: 20:14 monoclonal antibody (1:10 dilution) against the known human PAP gene: three isoforms with apparent MWs 90, 100, 106 kDA. This monoclonal antibody is directed against an epitope with the region of PAPγ that is shared between PAPs originating from the two different PAP genes. Lane 2, 3 polyclonal sera (dilution 1:2000, 1:4000 respectively) raised against a polypeptide comprising the C-terminal part located in the unique region starting at approximately amino acid 505 of PAPγ for specific recognition of one isoform, 90 kDa. Examples of such unique polypeptide is a polypeptide comprising amino acids located from amino acid 521 to the C-terminal end of PAPγ. This novel antibody is specific for PAPγ. This novel antibody was developed against the unique C-terminal part-of PAPγ. Below a detailed description of its production can be found. Lane 4: pre-immune serum (1:2000 dilution)—no detected signal.

Cloning of PAPγ in different cloning vectors for expression in prokaryotic systems

1.Cloning in pET-32a(+) vector

The coding sequence of PAPγ was amplified by PCR using cDNA library derived from HeLa total RNA by reverse transcription. The primers used to amplify the first 1479 nt from 5′part were the following: primer (a) (5′-CACCATGGAAGAGATGTCTGCAAACACC-3′) introducing a NcoI site(in italics) upstream of the initiation codon and primer (d) (5′-GAGAGCTCTTAGGTACCGTGAAGTTGTTTTTTCTTTACATGAGTTGC introducing a SacI site downstream of the stop codon; (for cloning reasons to the pCAL-c vector that will be described further on, we had to introduce a KpnI restriction site simultaneously, which introduces 2 extra aa at the C-terminus in all the pET-32a clones expressing PAPγ. The NcoI cloning site introduces a point mutation at the second aa in the sequence by changing Lys to glutamate)). The PCR product was cloned into pGEM-T vector and then by digestion NcoI/SacI and ligation was inserted to the pET-32a (NcoI/SacI digested); the clone is named pET-32(H1-493)—(where H denotes that the tag is N-terminally located and the numbers 1-493 refer to a polypeptide segment starting at amino acid 1 and ending at amino acid 493 of the human PAP sequence). Another pair of primers were used to amplify by PCR a fragment reaching up to 2208 nt 3′ of PAPγ; internal primer (c) 5′-GCCTGTCTGGGATCCTCGGGT-3′ and primer (g) (5′-GAGAGCTCTAAGGTACCTTTTCTTTTTCTTTCTTCAGCAGTGCG-3′). PCR product was cloned in pGEM-T, digested by EcoRI/SacI and inserted between the EcoRI and SacI restriction enzyme sites of plasmid pPAPγ(H1-493); the resulting clone is named pPAPγ(H1-683). For determination of the full length of PAPγ sequence 3′RACE methodology was performed according to “Clontech Smart Race cDNA amplification kit” using two upstream specific primers: primer (h) (CAACACCTCACAACCCTGCCCA) and primer (i) (GAGATCCCATTCCCCATCCATAG). The PCR product after seminested was cloned into pGEM-T vector and sequenced for confirmation of the identification of stoping codon. The new full-length 3′end of PAPγ was cloned by a new round of PCR amplification using the pGEM-T vector insert and the primer pairs (c) and primer (j) (5′-GAGAGGTACCAAGCCGATTAAGGGTCAGTCG). The new 3′end was cloned into the same starting clone pPAPγ(H1-493) by digestion EcoRI/SacI. The pET-32(a) vector introduces N-terminally of the gene of PAPγ a thyrodoxine tag which increases the expression of the soluble fraction of the recombinant protein, a histidine tag and an S-tag for enabling easy purification via affinity chromatography. The tag is about 20 kDa.

2. Cloning in pCAL-c vector.

The same cloning strategy was used but in the restriction cut the pair EcoRi/KpnI was used. The pCAL-c vector introduces a 4 kD calmodulin peptide tag which can be used for affinity purification with calmodulin-affinity resin and results in clones named pPAPγ(1-736C) or similarly where the numbers indicate encoded PAPγ amino acids while the C after the numbers denote a C-terminally located tag.

Expression and purification of recombinant form of PABγ cloned in PET32a vector, in E.coli.

PAPγ containing plasmids were used to transform BL21(DE3)pLysS E.coli strains. 1 colony is inoculated in 100 ml TB medium (containing phosphate) in the presence of 50 ug/ml carbenicillin and 34 μg/ml chloramphenicol and let grow by standing at 37° C. The 100 ml culture is inoculated in a final 1 lt culture in TB medium containing the required antibiotics. Bacteria are growing by vigorous shaking at 37° C. and are induced at OD₆₀₀ around 0.5-1.0 with 0.42 mM IPTG plus 0.524 mM MgCl₂. Cells where harvested by centrifugation 3 hours post inducion and pellets frozen at −70° C.

Extracts were made by thawing the cells on ice and lysing in 50 ml lysis buffer (20 mM Hepes/KOH pH7.5, 0.5M KCl, 1.0% NP-40, 1.0% Tween-20, 10% glycerol, 5 mM imidazole, 20 mM β-mercaptoethanol plus 1 tablet of EDTA-free protease inhibitors.); next follows sonication (3×10 sec), centrifugation 20 min at 39000 g and 0.45 μm filtration. The cell extracts are mixed batchwise to 1 ml Talon resin (Co++ affinity agarose) equilibrated in lysis buffer and proteins bound by 1 hr rotation at 4° C. The resin is packed in a manual chromatographic column and washed with 20 column volumes of lysis buffer; subsequently it is washed with 20 volumes wash buffer (lysis buffer without detergents and β-mercaptoethanol). The proteins are eluted by 5 volumes of elution buffer (20 mM Hepes/KOH pH7.5, 0.5M KCl, 10% glycerol, 200 mM imidazole, 0.5M KCl). The eluate is loaded in 1 ml HiTrap chelating column equilibrated in 20 mM Hepes/KOH pH7.5, 0.5M KCl, 1.0% NP-40, 1.0% Tween-20,10% glycerol, 50 mM imidazole). The column is washed with 10 column volumes equilibration buffer and 10 volumes wash buffer (equilibration buffer without detergents and containing 0.05 M KCl ). The proteins are eluted with 5 volumes elution buffer (20 mM Hepes/KOH pH 7.5, 0.05M KCl,10% glycerol, 0.5 mM DTT, 1.5 mM MgCl2, 200 mM imidazole). The eluate is loaded to a Heparin Hi-Trap column equilibrated in 20 mM Hepes/KOH pH 8.6, 0.05M KCl, 10% glycerol, 0.5 mM DTT, 1.5 mM MgCl2 buffer. It is washed with 5 volumes of the equilibration buffer and recombinant proteins eluted in fractions of 0.5 ml with 5 volumes of the same buffer containing 0.5 M KCl. In all buffer solutions they are added freshly 0.5 mM PMSF, 1.0 μg/ml leupeptin, 1.0 mg/ml pepstatin, 1.0 μg/ml aprotinin.

Expression and purification of recombinant form of PABγ cloned in pCAL-c vector, in E.coli.

PCAL-c PAPγ containing plasmids were used to transform BL21 (DE3)pLysS E.coli strains. 1 colony is inoculated in 50 ml TB medium (containing phosphate buffer) plus 50 μg/ml carbenicillin and let grow by standing at 37° C. The 50 ml culture is inoculated in a final 500 ml culture in TB medium containing antibiotics. Bacteria are growing by vigorous shaking at 37° C. and induced at OD₆₀₀ around 0.6-1.0 with 0.42 mM IPTG plus 0.524 mM MgCl₂. Cells where harvested by centrifugation 3 hours post induction and pellets frozen at −70° C. Extracts where made by unthawing the cells on ice and lysing in 30 ml lysis buffer—(Ca-binding buffer) (50 mM Tris/HCl pH7.5, 0.15 M KCl, 0.1 % Triton X-100, 10% glycerol, 1 mM Mg(CH₃COO), 2 mM CaCl₂, 1 mM imidazole, 10 mM β-mercaptoethanol plus 1 tablet of EDTA-free protease inhibitors.); next follows sonication (4×30 sec), centrifugation 20 min at 39000 g and 0.45 μm filtration. The cell extracts are mixed batchwise to 0.75 ml calmodulin resin (affinity agarose) equilibrated in binding buffer and proteins bound by rotation at 4° C. overnight. The resin is packed in a manual chromatographic column and washed with 20 column volumes of wash buffer I (50 mM Tris/HCl pH7.5, 0.2 M KCl, 0.1 % Triton X-100, 10% glycerol, 1 mM Mg(CHECOO), 2 mM CaCl₂, 1 mM imidazole, 10 mM β-mercaptoethanol;subsequently it is washed with 20 volumes washII buffer (50 mM Tris/HCl pH7.5, 0.25 M KCl,10% glycerol, 1 mM. Mg(CH₃COO), 2 mM CaCl₂, 1 mM imidazole). The recombinant protein is eluted in fractions of 0.5 ml with 7 volumes of buffer containing 50 mM Tris/HCl pH 7.5, 1 M KCl, 2 mM EGTA, 10% glycerol, 0.5 mM DTT and 1.5 mM MgCl₂. In all buffer solutions they are added freshly 0.5 mM PMSF, 1.0 μg/ml leupeptin, 1.0 mg/ml pepstatin, 1.0 μg/ml aprotinin.

Production of polyclonal sera recognizing specifically PAPγ

A unique part of the PAPγ sequence, as represented by a polypeptide starting at amino acid 521 and ending at amino acid 683, was cloned in pET-32(a) vector and the recombinant polypeptide was purified and used for immunization of rabbits. The 491 nt long fragment, comprising the above mentioned amino acids, was amplified using as template the plasmid pET-32(668) and a pair of primers: primer (p) (5′-CACCATGGAATCCAAAAGATTGTCTCTGGATAGC-3′) and primer (g) (5′-GAGAGCTCTTAGGTACCTTATTTTCTTTTTCTTTCTTCAGCAGTGCG-3′). The PCR product is cloned to pGEM-T vector and inserted to pET32(a) vector after restriction digestion with NcoI/BamHI. The recombinant polypeptide is expressed and purified, as described essentially, at the recombinant proteins' purification schedule. The antigen was more than 95% pure, was diluted to 0.9 mg/ml protein concentration. 500 μl of antigen were used for injection of 2 independent rabbits in repetitive injections.

### REFERENCES

Ballantyne, S., Bilger A., \dot{A}ström, J., Virtanen, A. and Wickens, M. (1995) Poly(A) polymerases in the nucleus and cytoplasm of frog oocytes: Dynamic changes during oocyte maturation and early development. RNA, 1, 64-78.

Colgan, D. F. and Manley, J. L. (1997) Mechanism and regulation of mRNA polyadenylation. Genes Dev., 11, 2755-2766.

Colgan, D. F., Murthy, K. G. K., Prives, C. and Manley, J. L. (1996) Cell-cycle related regulation of poly(A) polymerase by phosphorylation. Nature, 384, 282-285.;

Kyriakopoulou, C., Nordvarg, H., Virtanen, A. (2001). A novel nuclear human poly(A) polymerase, PAPγ. J.Biol.Chem. 276, 33504-33511.

Mitchell, P. and Tollervey, D. (2000) mRNA stability in eukaryotes. Curr Opin Genet Dev, 10, 193-8.

Raabe, T., Bollum, F. J. and Manley, J. L. (1991) Primary structure and expression of bovine poly(A) polymerase. Nature, 353, 229-234.

Sachs, A. B. and Varani, G. (2000) Eukaryotic translation initiation: there are (at least) two sides to every story. Nat Struct Biol, 7, 356-61.

Scorilas A., Courtis N., Yotis J., Talieri M., Michailakis M., Trangas T. (1998) Poly(A) polymerase activity levels in breast tumour cytosols. J Exp Clin Cancer Res 1998 Dec;17(4):511-8.

Scorilas A., Talieri M., Ardavanis A., Courtis N., Dimitriadis E., Yotis J., Tsiapalis C. M., and Trangas T. (2000) Polyadenylate polymerase enzymatic activity in mammary tumor cytosols: A new independent prognostic marker in primary breast cancer. Cancer Research 60, 5427-5433.

Thuresson, A. -C., \dot{A}ström, J., \dot{A}ström, A., Grönvik, K. -O. and Virtanen, A. (1994) Multiple forms of poly(A) polymerase in human cells. Proc. Natl. Acad Sci. USA, 91, 979-983.

Wahle, E., Martin, O., Schiltz, E. and Keller, W. (1991) Isolation and expression of cDNA clones encoding mammalian poly(A) polymerase. Embo J. 10, 4251-7.

Wahle, E. and Ruegsegger, U. (1999) 3′-End processing of pre-mRNA in eukaryotes. FEMS Microbiol Rev, 23, 277-95.

Zhao, J., Hyman, L. and Moore, C. (1999) Formation of mRNA 3′ ends in eukaryotes: mechanism, regulation, and interrelationships with other steps in mRNA synthesis. Microbiol Mol Biol Rev, 63, 405-45.

Zhao, W. and Manley, J. L. (1996) Complex Alternative RNA processing generates an unexpected diversity of poly(A) polymerase isoforms. Mol. Cell. Biol., 16, 2378-2386w.

1. A human poly(A) polymerase γ (PAPγ) comprising an amino acid sequence defined by a combination of four cDNA clones whereby a first cDNA clone [pPAP_(H1-493)] contains a sequence between the positions as defined by primer 5′-CACCATGGAAGAGATGTCTGCAAACACC-3′ and primer 5′-GAGAGCTCTTAGGTACCGTGAAGTTGTTTTTTCTTTACATGAGTTGC, a second cDNA clone [pPAP_(H1-683)] contains a sequence between the positions as defined by primer 5′-CACCATGGAAGAGATGTCTGCAAACACC-3′ and primer 5′-GAGAGCTCTAAGGTACCTTTTCTTTTTCTTTCTTCAGCAGTGCG-3′, a third cDNA clone [full length of PAP_] contains a sequence between the positions as defined by primer 5′-CACCATGGAAGAGATGTCTGCAAACACC-3′ and primer GAGAGGTACCAAGCCGATTAAGGGTCAGTCG, a fourth cDNA clone [pPAP_(1-736C)] contains a sequence between the positions as defined by primer 5′-CACCATGGAAGAGATGTCTGCAAACACC-3′ and primer 5′-GAGAGGTACCAAGCCGATTAAGGGTCAGTCG

2. A nucleic acid sequence according to Sequence Listing Id No 1 encoding the amino acid sequence(s) according to claim 1 and variants of said nucleic acid sequence due to the degeneracy of the genetic code.

3. An amino acid sequence according to Sequence Listing Id No 2 encoded according to claim 1 and/or functional parts thereof having poly(a)polymerase (PAP) activity as well as homologous variants of said sequence

4. A vector comprising the nucleic acid sequence according to claim 2.

5. A host cell comprising the vector according to claim 4.

6. A method for production of recombinant PAPγ, comprising cultivating the host cell according to claim 5 in a suitable cultivation media; and recovering PAPγ from said media.

7. Use of the nucleic acid sequence according to claim 2 or parts thereof for detection of PAP related diseases or disorders.

8. Use according to claim 7, wherein a PAPγ specific part is used.

9. An antibody against PAPγ according to claim 1 which is selective for PAPγ and does not react with other PAPs.

10. Use of the antibody according to claim 9 for detection of PAPγ related diseases and disorders.

11. A reagent comprising PAPγ according to claim 1.
